(19) United States
(12) Patent Application Publication
(10) Pub. No.: US 2008/0152698 A1
(43) Pub. Date: Jun. 26, 2008

(54) HYDROGEL

(75) Inventors: Jochem Effing, Kelkheim-Fischbach (DE); Ralph Geiger, Felsberg (DE); Gema Garrigos Sistare, Barcelona (ES)

Correspondence Address: BRINKS HOFER GILSON & LIONE, P.O. BOX 10395, CHICAGO, IL, 60610, US

(73) Assignee: Paul Hartmann AG, Heidenheim (DE)

(21) Appl. No.: 12/022,239

(22) Filed: Jan. 30, 2008

Related U.S. Application Data
(63) Continuation of application No. PCT/EP2006/007308, filed on Jul. 25, 2006.

(30) Foreign Application Priority Data
Jul. 30, 2005 (DE) 10 2005 035 879.9

(51) Int. Cl. A61L 15/00; A61K 31/717; A61K 31/722

(52) U.S. Cl. 424/445; 514/55; 514/57

(57) ABSTRACT

A hydrogel comprises at least 50% by weight of water, at least one gel-forming polysaccharide, at least one acrylic acid derivative and one electrolyte mixture, the electrolyte mixture containing at least two different electrolytes.

### CROSS-REFERENCE TO RELATED APPLICATIONS

This application is a continuation of International Application No. PCT/EP2006/007308 filed on Jul. 25, 2006, which claims the benefit of DE 10 2005 035 879.9, filed Jul. 30, 2005. The disclosures of the above applications are incorporated herein by reference.

### FIELD

The present invention relates to a hydrogel, as well as to its manufacture and use in modern wound treatment.

### BACKGROUND

The statements in this section merely provide background information related to the present disclosure and may not constitute prior art.

The use of hydrogels as wound treatment agents has been known for some time. These products are characterized by a high water content and are particularly suited for use in moist wound treatment. Hydrogels are available as freeze-dried pads, transparent compresses, or amorphous gels in tubes and syringes.

Hydrogels for wound treatment are known in patent literature, for example, from European Patent EP 382 128 B1. This describes a cross-linked, wound secretion absorbing hydrogel, which is produced by cross-linking a natural gelification agent selected from the group of collagens, gelatins, pectins, and/or alginates with a copolymer derived from one or more vinyl carboxylic acids and at least one of their salts. In addition to the polymers that are cross-linked by means of a cross-linking agent, the hydrogel also contains a polyvalent alcohol as well as water or saline.

Another hydrogel used as a wound dressing is described in EP 8484 621 B1, and contains a “bacteriostatic agent.” The fibers are understood to be incorporated into the gel and produce cations for cross-linking the hydrogel. The hydrogel is also understood to have a viscosity of 20,000 to 1,000,000 cPs.

Furthermore, EP 987 019 A1 describes a hydrogel for treating wounds that features a composition that exists in semi-solid form. In addition to water and an antibiotic, the composition also contains particles that are able to absorb at least 30 percent by weight of water, and to release at least 70 percent by weight of water. Apart from a polyol, the semi-solid composition is understood to contain at least two gelling agents.

DE 100 12 026 A1 describes a gel, and the use of a gel to dissolve wound crusts. The described aqueous gel is understood to have a pronounced microbiocidic effect and contain polyhexamethyleneguanidine as well as glycerin and hydroxyethyl cellulose. In addition, the gel may feature a saline or Ringer's solution as an aqueous solution.

A hydrogel for use as a wound dressing is also described in the European patent specification EP 576 523 B2. This hydrogel consists of a cross-linked, water-insoluble, water-swellable cellulose derivative, water, and a polyol component. The gel described in this patent specification is primarily used for the removal of necrotic tissue because it reduces the necessity of using a chemical debriding agent or surgical excision.

These hydrogels, known from the state of the art, typically display overall or partial characteristics considered as a disadvantage by the user. For example, these hydrogels may contain substances that the user is quite critical of in some treatments, or that the user considers to disturb the progressing healing process.

### SUMMARY

The present invention provides a hydrogel having characteristics that a user typically considers more desirable for use in modern wound treatment. For example, the present disclosure provides a hydrogel that, when applied on a wound, creates an environment that promotes wound healing and avoids the disadvantages of known hydrogels. The hydrogel may be used on dry wounds, as well as on wounds excreting wound secretion, to provide gentle debridement in the treatment of these different wounds. Furthermore, the present disclosure provides a hydrogel with an analgesic effect, and which provides a balance between the wound and the wound treatment agent when applied. The hydrogel may also be used as a wound filler, which, in its applicable condition, may be easily molded, and may ensure good cohesion when absorbing wound exudate without appreciable degradation of viscosity. The present disclosure also provides a method of manufacturing a hydrogel that promotes wound healing.

In one form, the present invention provides a hydrogel with a water content of at least 50 percent by weight relative to the total weight of the hydrogel. The hydrogel contains at least one gel-forming polysaccharide, at least one acrylic acid derivative, and one electrolyte mixture that features at least two different electrolytes.

Depending on the type of wound, the combination of gel-forming polysaccharides and an acrylic acid derivative may have both an absorbent effect on strongly wetting wounds, as well as a hydrating effect on dry wounds, and may be easily molded. Surprisingly, it has been found that a hydrogel of this type may also be sterilized by means of electromagnetic radiation, or electron or positron radiation. If only gel-forming polysaccharides are used for hydrogel generation, a gel of inadequate viscosity and insufficient water-absorption capacity is obtained after sterilization by radiation or particle radiation. If, on the other hand, a gel is designed based on acrylic acid derivatives only, a gel is obtained that may be sterilized by radiation or particle radiation, but may be poorly molded, and which also has poor water releasing capacity. In addition, via the electrolyte mixture, a hydrogel according to the present invention may provide the wound with an environment that greatly promotes wound healing because with the at least two electrolytes, an electrolyte mixture similar to wound serum may be provided. Moreover, with this hydrogel, a wound treatment agent may be provided that breaks down necrotic tissue present in a wound and ensures gentle debridement. This debridement helps facilitate natural cell formation, which, beginning at the edges of the wound, characterizes a continuous healing process.

In another form, a wound filler is provided, which includes a hydrogel. The hydrogel has a water content of at least 50 percent by weight of the total weight of the hydrogel. The hydrogel includes at least one gel-forming polysaccharide, at least one acrylic acid derivative, and an electrolyte mixture. The electrolyte mixture includes at least two different electrolytes.

In yet another form, a wound dressing is provided. The wound dressing includes a drug carrier material and a hydrogel having a water content of at least 50 percent by weight of the total weight of the hydrogel. The hydrogel includes at least one gel-forming polysaccharide, at least one acrylic acid derivative, an electrolyte mixture. The electrolyte mixture includes at least two different electrolytes.

In still another form, a method for manufacturing a hydrogel is provided. The method includes preparing an aqueous solution that includes an acrylic acid derivative, adding a suspension having at least one polysaccharide in powder form to the aqueous solution to form a composition, and irradiating the composition with either electromagnetic radiation, electron radiation, or positron radiation, to adjust the viscosity of the hydrogel.

In still another form, a method of treating a wound is provided. The method includes applying a hydrogel to a wound, wherein the wound is one of the following: decubitus stage I, II, III (bedsore), crural ulcer (leg ulcers, open sores on the leg), diabetic foot syndrome, a skin ulcer, boils, a first-degree burn, a second-degree burn, skin abrasions, or a chronic wound. The hydrogel to be applied has a water content of at least 50 percent by weight of the total weight of the hydrogel and includes at least one gel-forming polysaccharide, at least one acrylic acid derivative, and an electrolyte mixture, wherein the electrolyte mixture includes at least two different electrolytes.

In still another form, a hydrogel for treating a wound is provided. The hydrogel has a water content of at least 50 percent by weight and includes an electrolyte mixture. The electrolyte mixture includes at least one of the following: sodium chloride, potassium chloride, and/or calcium chloride.

In still another form, another hydrogel for treating a wound is provided. The hydrogel has a water content of at least 50 percent by weight and a conductivity of at least 4000 μS·cm⁻.

Further areas of applicability will become apparent from the description provided herein. It should be understood that the description and specific examples are intended for purposes of illustration only and are not intended to limit the scope of the present disclosure.

### BRIEF DESCRIPTION OF DRAWINGS

The drawing described herein is for illustration purposes only and is not intended to limit the scope of the present disclosure in any way.

FIG. 1 is a graph illustrating conductivity as a function of electrolyte concentrations.

### DETAILED DESCRIPTION

The following description is merely exemplary in nature and is not intended to limit the present disclosure, application, or uses.

In one form, a hydrogel according to the present invention contains no preservatives. Furthermore, the hydrogel is also preferably free of antimicrobial, antifungal, antibacterial, and any other agents that in any way kill or inhibit the growth of fungi, microbes, bacteria or viruses.

In some preferred forms, the hydrogel advantageously contains at least one gel-forming polysaccharide. The gel-forming polysaccharide may be selected from the following group: cellulose derivatives or their salts, alginates or their derivatives, chitin or its derivatives, or its salts or starches. The origin of the gel-forming polysaccharide is irrelevant, i.e., this gel-forming polysaccharide may be of vegetable or animal origin, or may be produced synthetically, by microbiological processes, for example. It is also possible to use polysaccharides of vegetable or animal origin modified by chemical synthesis.

In connection with the present invention, the group of cellulose derivatives may include cellulose ethers and cellulose esters as well as their salts. One example of a cellulose ether that may be used is hydroxy alkyl cellulose, for example, hydroxy C₁₋₆-alkyl cellulose such as hydroxymethylcellulose, hydroxyethyl cellulose, hydroxy propyl cellulose, hydroxybutyl cellulose, or hydroxymethylcellulose or hydroxyethyl cellulose. One example of a cellulose ester that may be used is carboxy alkyl cellulose, particularly carboxy-C₁₋₆ alkyl cellulose, such as carboxymethylcellulose, carboxyethyl cellulose, carboxypropyl cellulose, carboxybutyl cellulose, or carboxymethylcellulose, or carboxyethyl cellulose.

In another form, the hydrogel may contain at least two different gel-forming polysaccharides. In some forms, the two polysaccharides may be selected from the following group: cellulose derivatives or their salts, for example cellulose ethers and cellulose esters, alginates or their derivatives, chitin or its derivatives, or its salts or starches. In some forms, the hydrogel may contain at least two polysaccharides from the group of cellulose ethers and cellulose esters, for example, the hydrogel may contain hydroxy alkyl cellulose and carboxy alkyl cellulose as a gel-forming polysaccharide.

Furthermore, a hydrogel according to the present invention may contain at least one water-soluble cellulose derivative as a gel-forming polysaccharide. In some forms, this hydrogel may contain at least two different gel-forming, water-soluble polysaccharides. The polysaccharides may be non cross-linked water-soluble cellulose derivatives, by way of example. The polysaccharides may be of the type that do not form any swelled particles inside the gel, which in turn results in a very homogenous hydrogel. In addition, a gel containing a water-soluble polysaccharide presents especially good spreadability when applied to a wound, forms a particularly smooth surface, and is particularly easy to mold.

In some variations, the first gel-forming polysaccharide may be a cellulose derivative, such as hydroxy alkyl cellulose or carboxy alkyl cellulose, and the second gel-forming polysaccharide may be selected from the following group: alginates, such as sodium, potassium, or calcium alginate, and/or chitin or its derivatives or salts.

When using two gel-forming polysaccharides, a first nonionic polysaccharide and at least a second ionic polysaccharide may be used. However, two nonionic polysaccharides or two ionic polysaccharides may alternatively be used. Examples of nonionic polysaccharides include water-soluble cellulose ether and water-soluble hydroxy alkyl cellulose. Examples of ionic polysaccharides include water-soluble cellulose esters, water-soluble alkyl celluloses, an alginate, or a mixture of different alginates, such as sodium or calcium alginate.

The acrylic acid derivative present in a hydrogel according to the present invention may be a structure-forming or viscosity-improving agent. Particularly suitable for this purpose are polyacrylic acids and their salts, and in particular cross-linked polyacrylates. These polyacrylic acid derivatives additionally feature the advantage that they may absorb a considerable percentage of their own weight in water. By combining these acrylic acid derivatives with at least one gel-forming polysaccharide, it is possible to manufacture a specific hydrogel, whose water absorption and water releasing capacities may be controlled.

Furthermore, the hydrogel may feature a dynamic viscosity of 5,000 to 60,000 mPa·s, in particular 5,000 to 50,000 mPa·s, and very particularly 10,000 to 40,000 mPa·s (measured with a Bohlin rheometer Type CSR-10, cone spindle 4°/Ø 40 mm, slit gap 100 μm, oscillometric measurement T=22-27° C.). This kind of hydrogel may be spread particularly well and evenly over and into a wound with a spatula for example, with good cohesion when absorbing wound exudate, and does not leak out of the wound that is being treated.

In one preferred form, the hydrogel contains at least one gel-forming polysaccharide and one acrylic acid derivative, the weight ratio of the polysaccharide or polysaccharides to the acrylic acid derivative in the hydrogel being from 20:1 to 1:1, preferably 15:1 to 1:1, and more preferably 10:1 to 1:1.

In one variation, the hydrogel contains at least 50 percent by weight of water, 0.5-10 percent by weight of gel-forming polysaccharide, 0.05-6.0 percent by weight of acrylic acid derivative, and 0.001-4.0 percent by weight of electrolyte mixture. In some variations, the hydrogel contains at least 50 percent by weight of water, 1-6 percent by weight of gel-forming polysaccharide, 0.5-4.0 percent by weight of acrylic acid derivative, and 0.001-2.0 percent by weight of electrolyte mixture.

If, apart from a first gel-forming polysaccharide, a second gel-forming polysaccharide is added to the hydrogel, this hydrogel may contain at least 50 percent by weight of water, 0.5-5 percent by weight of a first gel-forming polysaccharide, 0.5-5 percent by weight of a second gel-forming polysaccharide, 0.05-6.0 percent by weight of an acrylic acid derivative, and 0.001-4.0 percent by weight of an electrolyte mixture. In some variations, the hydrogel may contain at least 50 percent by weight of water, 0.5-4 percent by weight of a first gel-forming polysaccharide, 0.5-4 percent by weight of a second gel-forming polysaccharide, 0.5-4.0 by weight of an acrylic derivative, and 0.001-2.0 percent by weight of an electrolyte mixture.

If at least two different gel-forming polysaccharides are used, the weight ratio of the first to the second polysaccharide may be from 1:6 to 6:1, and preferably 1:4 to 4:1. If hydroxyethyl cellulose is used as a first gel-forming polysaccharide, for example, and carboxymethylcellulose is used as a second gel-forming polysaccharide, the ratio of these two components may be used to determine the water absorbing and/or water releasing capacity of the gel. If the percentage of carboxymethylcellulose is set equal to one, for example, and the percentage of hydroxyl ethyl cellulose is set greater than one, a hydrogel will be obtained that exhibits greater water release compared to a hydrogel that contains the same percentages of the two cellulose derivatives. If, on the other hand, the percentage of hydroxyalkyl cellulose is set equal to one, and the percentage of carboxymethylcellulose is set equal to greater than one, a hydrogel will be obtained that exhibits higher water absorption compared to a hydrogel that contains the same percentage of the two cellulose derivatives. The testing of the water absorption and/or water releasing capacity is performed analogous to the testing described in St. Thomas and P. Hay Ostomy/Wound Management 1995, Vol. 41, no. 3, pp. 54-59.

In one variation, the hydrogel may be provided as a sterilized hydrogel. For example, the hydrogel may be sterilized by electromagnetic radiation or electron or positron radiation. However, the hydrogel may also or alternatively be sterilized by means of steam sterilization, or any other suitable method.

Sterilizing by means of electromagnetic radiation or electron or positron radiation may provide a hydrogel having a dynamic viscosity ranging from 5,000 to 60,000 mPa·s, preferably 5,000 to 40,000 mPa·s, and more preferably 10,000 to 40,000 mPa·s (Bohlin rheometer type CSR-10, cone spindle 4°/Ø 40 mm, slit gap 100 μm oscillometric measurement). This kind of hydrogel may be spread well and evenly over and into a wound with a spatula for example. This hydrogel also shows good cohesion when absorbing wound exudate, and does not leak out of the wound that is being treated.

Suitable electrolytes in connection with the present invention are compounds that are able to dissociate into ions, particularly when dissolved in water, and which are composed of monovalent, divalent, and/or trivalent ions. These electrolytes may be found as inorganic or organic salts, for example, and in each case are different from the polymers that are likewise contained in the present hydrogel and have a potentially ionic character. Particularly suitable in this regard are chlorides, iodides, sulfates, hydrogen sulfates, carbonates, hydrogen carbonates, phosphates, dihydrogen phosphates, or hydrogen phosphates of the alkali and alkaline earth metals. Sodium, potassium, and calcium chloride in particular may be used as an electrolyte mixture in a hydrogel according to the present invention. This electrolyte mixture simulates the electrolyte mixture in the wound serum that is excreted by a wound especially well. A hydrogel containing this electrolyte mixture therefore provides the wound with an environment that especially promotes wound healing.

In one variation, the hydrogel features a conductivity of at least 4000 μS cm⁻¹, preferably at least 6000 μS cm⁻¹ and more preferably 6000-20,000 μS cm⁻¹. The conductivity may be adjusted, among other ways, via the amount of electrolyte mixture. This adjustment is advantageous because the conductivity of a hydrogel depends on the components of the gel and their concentrations. The conductivity depends, for example, on the type and concentration of the gel-forming polymers used or on the type and concentration of the polyols used. These components reduce the conductivity of a hydrogel to different extents compared to pure saline solution. The amount of electrolyte mixture should therefore be adjusted individually to the rest of the ingredients of the gel in order to adjust a defined conductivity.

In some forms, the hydrogel features an amount of electrolytes such that the hydrogel itself has a free ion concentration corresponding to the free ion concentration of a physiological electrolyte solution, and/or isotonic electrolyte solution. Alternatively, the hydrogel may be an isotonic hydrogel.

In one variation, the hydrogel may contain a Ringer's solution. A Ringer's solution should be understood as an isotonic saline solution containing sodium chloride, potassium chloride, and calcium chloride.

In yet another variation, the hydrogel features a polyol. This polyol is preeminently suitable as a moisturizer, and therefore is a conditioning component for the skin surrounding the wound. The polyol may be glycerin, glycol, propylene glycol, polyethylene glycol, polypropylene glycol, polyethylene propylene glycol, sorbitol, and their compounds, by way of example.

If a polyol is added to the hydrogel, the hydrogel may contain at least 50 percent by weight of water, 5-30 percent by weight of polyol, 0.5-10 percent by weight of gel-forming polysaccharide, 0.05-6.0 percent by weight of an acrylic acid derivative, and 0.001-4.0 percent by weight of an electrolyte mixture. In some variations, the hydrogel may feature at least 50 percent by weight of water, 10-30 percent by weight of polyol, 1-6 percent by weight of gel-forming polysaccharide, 0.5-4.0 percent by weight of acrylic acid derivative, and 0.001-2.0 percent by weight of electrolyte mixture.

If, in addition to a polyol and a first gel-forming polysaccharide, a second gel-forming polysaccharide is added to the hydrogel, the hydrogel may contain at least 50 percent by weight of water, 5-30 percent by weight of polyol, 0.5-5 percent by weight of the first gel-forming polysaccharide, 0.5-5 percent by weight of the second gel-forming polysaccharide, 0.05-6.0 percent by weight of an acrylic acid derivative, and 0.001-4.0 percent by weight of an electrolyte mixture.

In some variations, the hydrogel may feature at least 50 percent by weight of water, 10-30 percent by weight of polyol, 0.5-4 percent by weight of a first gel-forming polysaccharide, 0.5-4 percent by weight of a second gel-forming polysaccharide, 0.5-4.0 percent by weight of an acrylic acid derivative, and 0.001-2.0 percent by weight of an electrolyte mixture.

In addition to the hydrogel itself, the manufacture of a hydrogel, in particular a hydrogel as described above is also the subject matter of the present invention. In one form, the method comprises the following process steps:
a) Preparation of an aqueous solution containing an acrylic acid derivative;
b) Addition of a suspension containing at least one polysaccharide in powder form to the solution prepared according to a) above; and
c) irradiation of the mixture produced according to b) above with electromagnetic radiation or electron or positron radiation in order to adjust the viscosity of the hydrogel.

According to a further development of the method, the aqueous solution containing an acrylic acid derivative prepared according to a) above is adjusted to a pH value between 5.5 and 7.0. In one variation, the suspension mentioned in b) above also contains a polyol, in particular a liquid polyol. Furthermore, this suspension is preferentially prepared by stirring the polysaccharide in powder form into a polyol in liquid form at room temperature. The suspension added according to b) above may be produced by stirring at least one polysaccharide into a liquid polyol at room temperature, and adjusting the aqueous solution mentioned in a) above to a pH value between 5.5 and 7.0 before adding the suspension manufactured according to b) above. Further, the aqueous solution mentioned in a) above may contain an electrolyte mixture that on its part contains at least two electrolytes. Furthermore, the irradiation may be carried out by means of β-radiation. Doses of 20-35 kGy are particularly advantageous.

In another form, a composition is provided, which is suitable for producing a hydrogel that is sterilized by means of electromagnetic radiation or electron or positron radiation. The composition contains at least one gel-forming polysaccharide, one acrylic acid derivative, and an electrolyte mixture that contains at least two different electrolytes and has a dynamic viscosity not exceeding 40,000 mPa·s (measured with a Bohlin rheometer type CSR-10, cone spindle 4°/Ø 40 mm, slit gap 100 μm, oscillometric measurement, T=22-27° C.).

A hydrogel according to the present invention is particularly suitable for treating a comparatively deep wound, and is outstandingly well suited for use as wound filler. Deep dermal ulcers for example, which very often wet strongly, may be treated with this hydrogel. The seeping of fluid from the wound is prevented, or at least reduced, and at the same time, by providing an electrolyte mixture, a wound treatment agent is also provided that promotes wound healing. Furthermore, dry wounds, such as dry crural ulcers for example, may also be treated with this gel. The present hydrogel then demonstrates its ability to provide the wound with fluid and ensure the removal of undesirable substances, layers and necroses by means of gentle debridement. The wound healing process is assisted by a semi-occlusive seal by means of a secondary wound dressing, such as a foil dressing for example, by means of which undesirable contamination may be prevented. Other wound categories for which the gel may be used, without being restricted to them, include decubitus stage I, II, III (bedsore), crural ulcers (leg ulcers, open sores on the leg), diabetic foot syndrome, skin ulcers, boils, first and second-degree burns, skin abrasions, and chronic wounds. The present disclosure therefore relates to the use of a hydrogel that contains at least one gel-forming polysaccharide, one acrylic acid derivative, and one electrolyte mixture, the electrolyte mixture featuring at least two different electrolytes for the manufacture of a wound healing agent, in particular for the treatment of decubitus stage I, II, III (bedsore), crural ulcers (leg ulcers, open sore on the leg), or diabetic foot syndrome, or skin ulcers, or boils, or first and second-degree burns, or skin abrasions, or chronic wounds.

In addition to the hydrogel as such, the present disclosure relates to a wound dressing that contains a drug carrier material and a hydrogel of the described type. As carrier materials, nonwoven or knit fabrics, knitted or woven fabrics made of natural or synthetic fibers are used. In particular, the drug carrier material is coated or impregnated with the hydrogel on one or more sides.

In another form, the hydrogel may be arranged in a package. More particularly, the hydrogel may be sterile packaged. In these cases, packages such as containers with screw caps, reclosable tubes, or expendable containers such as tubes with safety caps, for example, may be used. In another variation, the hydrogel may be arranged in a syringe used as the original package. More particularly, the hydrogel in the syringe may be sterile. In some forms, this hydrogel may be contained in a sterile original package as a ready-for-use kit and may be available together with a drug carrier, dressing material, or medical aid. Both the hydrogel in the original package and the drug carrier or dressing materials may be available in a sterile original package in the kit package.

### EXAMPLES

### 1)Examples 1 to 4

### a) Composition, Examples 1 to 4

| | Hydrogel | | Hydrogel | | Hydrogel | | Hydrogel | |
|---|---|---|---|---|---|---|---|---|
| | 1 | | 2* | | 3 | | 4 | |
| Hydroxy ethyl | 300 | g | 300 | g | 300 | g | 300 | g |
| cellulose⁽¹⁾ | | | | | | | | |
| Carboxy- | | | 100 | g | 100 | g | 100 | g |
| methylcellulose⁽²⁾ | | | | | | | | |
| Polyacrylate⁽³⁾ | 70 | g | 70 | g | 70 | g | 70 | g |
| Glycerin⁽⁴⁾ | 2000 | g | 2000 | g | 2000 | g | 2000 | g |
| Sodium chloride (p.a.) | | | | | | | 84 | g |
| Calcium chloride | | | | | | | 3.1 | g |
| dihydrate (p.a.) | | | | | | | | |
| Potassium chloride | | | | | | | 2.9 | g |
| (p.a.) | | | | | | | | |
| 1N sodium hydroxide | 750 | g | 750 | g | 750 | g | 750 | g |
| solution (p.a.) | | | | | | | | |
| Water, purified, Ph. Eur. | | | 6780 | g | | | 6690 | g |
| Ringer's solution⁽⁵⁾ | 6880 | g | | | 6780 | g | | |

| | | | | |
|---|---|---|---|---|
| pH value^{(A)} | 6.0 | 6.0 | 6.0 | 6.0 |
| Viscosity^{(B)}/Pa s: | | | | |
| a) non-sterile | 120 | 195 | 190 | 200 |
| b) sterile^{(D)} | | | 27 | 22 |
| Conductivity^{(C)}/μS cm⁻¹ | | | | |
| a) non-sterile | 7460 | 1456 | 8029 | 10300 |
| b) sterile^{(D)} | | | | 10733 |

*Not a hydrogel according to the present invention

| | |
|---|---|
| ⁽¹⁾ Hydroxyethyl cellulose (HEC) | Natrosol HX Pharm |
| | (manufacturer: Hercules, Rijswijk - |
| | Netherlands) |
| ⁽²⁾Carboxymethylcellulose (CMC) | Blanose 7H4 |
| | (manufacturer: Hercules, Rijswijk - |
| | Netherlands) |
| ⁽³⁾Polyacrylate | Carbopol 980 NF |
| | (manufacturer: Noveon, Calvert |
| | City - USA) |
| ⁽⁴⁾Glycerin (water-free) | (manufacturer: DOW Deutschland |
| | Inc.) |
| 13/19 | |
| ⁽⁵⁾Ringer's solution: | 8.60 g sodium chloride (NaCl) |
| 1000 ml of solution contains: | 0.30 g potassium chloride (KCl) |
| | 0.33 g calcium chloride dehydrate |
| | (CaCl₂ * 2H₂O) |
| | Rest: water, pure Ph. Eur. (H₂O) |

^{(A)}The pH value is determined with a pH meter type CG 841
(manufacturer: Schott - Germany) equipped with a glass electrode SenTix
81 (manufacturer: Technische Werkstatten GmbH, Weilheim - Germany).
The samples are tempered before measurement at 25° C. and measured at
25° C. room temperature.
^{(B)}The viscosity is measured with a Bohlin rheometer type CSR - 10
(F. Bohlin Instruments, Muhlacker - Germany), cone spindle 4°/Ø 40 mm,
slit gap 100 μm, oscillometric measurement. The samples are tempered at
25° C. before measurement and measured at 25° C.
^{(C)}The conductivity is measured with a standard conductivity measuring
cell Tetracon 325 (Manufacturer: Wissenschaftlich-Technische Werstatten
GmbH, Weilheim - Germany). The samples are tempered at 25° C. before
measurement and measured at 22-25° C.
^{(D)}The sterile hydrogels are sterilized using β-radiation. Depending on the
irradiation arrangement of the gel samples, the dose distribution is between
25 to 36 kGy. In order to achieve a low dose distribution, the 30-gram gel
samples are filled into 55-ml plastic containers (diameter 55 mm, material
PP). A minimum irradiation of 25 kGy was applied as sterile.

### b) Manufacture of Hydrogels 1 to 4

The hydrogels 1 to 4 are all manufactured at room temperature according to the specified production steps. In order to produce the hydrogels, a suspension of powdered polysaccharide (HEC or a mixture of HEC and CMC) and glycerin is manufactured in a first step, while the polysaccharide is slowly added to the glycerin and stirred continuously. In a second step, a solution of the polyacrylate is manufactured. For this purpose, the amount of powdered polyacrylate specified above is added to the Ringer's solution, to water, or to the aqueous electrolyte solution, and stirred for two hours. By adding the sodium hydroxide solution, a pH value=6 is set and stirred for a further two hours. Subsequently, the suspension of HEC/glycerin or HCE/CMC/glycerin is added very slowly to the solution adjusted to pH=6 stirring constantly. After this addition, stirring is continued for at least two hours at room temperature. During manufacture, air may be incorporated into the gel. These inclusions may be removed by vacuum stirring. The resulting hydrogels are filled into tubes and sterilized by β-radiation. Depending on the irradiation arrangement of the gel samples, the dose distribution is between 25 to 36 kGy. The viscosity of the hydrogel is adjusted to approximately 1/10 of the viscosity of non-sterile hydrogel by the irradiation.

### c) Description of the Hydrogels

The resulting hydrogels 1 to 4 (the hydrogel 2 is not a hydrogel according to the claims) are all transparent, amorphous hydrogels with good to very good plasticity. The hydrogels 3 and 4 thus have a high viscosity in a non-sterile state and a lower viscosity (approx. 1/10 of the non-sterile hydrogels) in a sterile state. Furthermore, especially with hydrogel 1, 3, and 4, a hydrogel for treating wounds may be manufactured that has an electrolyte composition similar to that of wound serum. The changes in conductivity between non-sterile and sterile gel are within the measuring tolerances.

### 2)Examples 5 to 8

### a) Composition, Example 3 and 5 to 8

The hydrogels 3, 5 to 8 have all he same composition as that specified for hydrogel 3, except that a solution similar to a Ringer's solution is used as a Ringer's solution.

| | Concentration of the solution | Conductivity (non- |
|---|---|---|
| | similar to a Ringer's solution^{(E)} | sterile) |
| Ringer's solution | | 16150 μS cm⁻¹ |
| Hydrogel 5 | x = 0.5 | 4120 μS cm⁻¹ |
| Hydrogel 3 | x = 1.0 = Ringer's solution | 8020 μS cm⁻¹ |
| Hydrogel 6 | x = 1.5 | 11140 μS cm⁻¹ |
| Hydrogel 7 | x = 2.0 | 12620 μS cm⁻¹ |
| Hydrogel 8 | x = 2.5 | 16370 μS cm⁻¹ |

^{(E)}1000 ml solution of a solution similar to a Ringer's solution contains in each case:
x times 8.60 g sodium chloride (NaCl)
x times 0.30 g potassium chloride (KCl)
x times 0.33 g calcium chloride dihydrate (CaCl₂ * H₂O)
rest: water, purified Ph. Eur. (H₂O)

### b) Manufacture of Hydrogels 5 to 8

The hydrogels 5 to 8 are manufactured analogous to the hydrogels 1 to 4

### c) Description of the Hydrogels

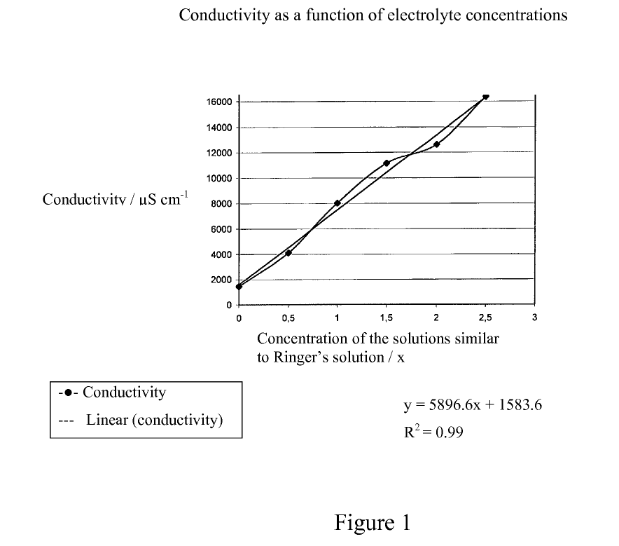

The hydrogels 3, 5 to 8 contain the composition specified for the example of hydrogel 3, with the exception that instead of a Ringer's solution, a solution analogous to a Ringer's solution is used. These solutions are different from one another only in the content of electrolyte mixture. Compared to hydrogel 3, the hydrogel 7 features twice the amount of electrolyte mixture, for example. The conductivity of the various gels differs considerably. In particular, hydrogel 3, which contains Ringer's solution, only has a conductivity that is half as high as the actual Ringer's solution. A conductivity analogous to that of Ringer's solution is only achieved with the hydrogel 8. This hydrogel 8 features 2.5 times the amount of electrolyte mixture compared to hydrogel 3. The hydrogel 8 may be characterized as an isotonic hydrogel, as this hydrogel has the same conductivity as an isotonic Ringer's solution. The conductivity is measured in a non-sterile state. The rise in conductivity is approximately linear, as may be seen from FIG. 1. This also shows that once a composition has been defined, the conductivity may be controlled by varying the electrolyte amount.

It should be noted that the disclosure is not limited to the embodiment described and illustrated as examples. A large variety of modifications have been described and more are part of the knowledge of the person skilled in the art. These and further modifications as well as any replacement by technical equivalents may be added to the description and figures, without leaving the scope of the protection of the disclosure and of the present patent.

What is claimed is:

1. A hydrogel with a water content of at least 50 percent by weight of the total weight of the hydrogel, the hydrogel comprising:
at least one gel-forming polysaccharide;
at least one acrylic acid derivative; and
an electrolyte mixture, wherein the electrolyte mixture comprises at least two different electrolytes.

2. The hydrogel according to claim 1, the hydrogel having a conductivity of at least 4000 μS·cm⁻¹.

3. The hydrogel according to claim 1, the at least one gel-forming polysaccharide being at least two gel-forming polysaccharides, the at least two gel-forming polysaccharides being at least two different gel-forming polysaccharides.

4. The hydrogel according to claim 1, the at least one polysaccharide being selected from the group consisting of: hydroxy celluloses, carboxy celluloses, alginates, alginate derivatives, chitin, chitin derivatives, chitin salts, and chitin starches.

5. The hydrogel according to claim 1, the at least one gel-forming polysaccharide being at least two gel-forming polysaccharides, the at least two gel-forming polysaccharides being cellulose derivatives.

6. The hydrogel according to claim 1, the at least one polysaccharide being a water-soluble, non-cross-linked, cellulose derivative.

7. The hydrogel according to claim 1, the at least one acrylic acid derivative being a cross-linked polyacrylate.

8. The hydrogel according to claim 1, the weight ratio of the at least one polysaccharide to the at least one acrylic acid derivative being in the range of about 20:1 to 1:1.

9. The hydrogel according to claim 1, the hydrogel being sterilized by one of electromagnetic radiation, electron radiation, and positron radiation.

10. The hydrogel according to claim 1, further comprising a polyol, the polyol being selected from the group consisting of: a glycerin, sorbitol, and polyethylene glycol.

11. The hydrogel according to claim 1, the hydrogel having a pH value between about 5 and 7.

12. The hydrogel according to claim 1, the hydrogel having a dynamic viscosity in the range of about 5000 to 60,000 mPa·s.

13. A wound filler comprising a hydrogel, the hydrogel having a water content of at least 50 percent by weight of the total weight of the hydrogel, the hydrogel comprising:
at least one gel-forming polysaccharide;
at least one acrylic acid derivative; and
an electrolyte mixture, wherein the electrolyte mixture comprises at least two different electrolytes.

14. A wound dressing comprising:
a drug carrier material; and
a hydrogel having a water content of at least 50 percent by weight of the total weight of the hydrogel, the hydrogel comprising:
at least one gel-forming polysaccharide;
at least one acrylic acid derivative; and
an electrolyte mixture, wherein the electrolyte mixture comprises at least two different electrolytes.

15. The wound dressing according to claim 14, the drug carrier material being impregnated with hydrogel.

16. A method for manufacturing a hydrogel, the method comprising:
preparing an aqueous solution comprising an acrylic acid derivative;
adding a suspension comprising at least one polysaccharide in powder form to the aqueous solution to form a composition; and
irradiating the composition with one of electromagnetic radiation, electron radiation, and positron radiation, to adjust the viscosity of the hydrogel.

17. The method according to claim 16, further comprising adjusting the aqueous solution to a pH value between about 5.5 and 7 before adding the suspension.

18. A method of treating a wound, the method comprising applying a hydrogel to a wound, the wound being one of decubitus stage I, decubitus stage II, decubitus stage III, crural ulcer, diabetic foot syndrome, a skin ulcer, boils, a first-degree burn, a second-degree burn, skin abrasions, and a chronic wound, the hydrogel having a water content of at least 50 percent by weight of the total weight of the hydrogel, the hydrogel comprising:
at least one gel-forming polysaccharide;
at least one acrylic acid derivative; and
an electrolyte mixture, wherein the electrolyte mixture comprises at least two different electrolytes.

19. A hydrogel for treating a wound, the hydrogel having a water content of at least 50 percent by weight, the hydrogel comprising an electrolyte mixture, the electrolyte mixture comprising at least one of sodium chloride, potassium chloride, and calcium chloride.

20. A hydrogel for treating a wound, the hydrogel having a water content of at least 50 percent by weight, the hydrogel having a conductivity of at least 4000 μS·cm⁻¹.
